(19) United States
(12) Patent Application Publication
(10) Pub. No.: US 2026/0273149 A1
(43) Pub. Date: Sep. 17, 2026

(54) GRAVITY BASED DIALYSIS SYSTEM AND DEVICE AND METHOD FOR THERAPY PRESCRIPTION OPTIMISATION

(71) Applicant: Vivatum PD AG, Eschenbach (CH)

(72) Inventors: David THORNE, Eschenbach (CH); Evan SMIT, Eschenbach (CH)

(73) Assignee: Vivatum PD AG, Eschenbach (CH)

(21) Appl. No.: 19/162,446

(22) PCT Filed: Mar. 6, 2024
(86) PCT No.: PCT/IB2024/052171

§ 371 Date: Sep. 5, 2025

(30) Foreign Application Priority Data
Mar. 6, 2023 (GB) 2303239.4

(51) Int. Cl. A61M 1/28

(52) U.S. Cl. CPC A61M 1/28; A61M 2205/3306; A61M 2205/3344; A61M 2205/3393; A61M 2205/502; A61M 2205/52

(57) ABSTRACT

Apparatus and methods are provided for performing continuous ambulatory peritoneal dialysis (CAPD) on a patient using a device (10) with a user interface (28) and programmable circuitry on which prescription data is entered. Information according to the prescription is provided to the patient on the user interface (28), and treatment data relating to administered CAPD are gathered and communicated to a healthcare provider. Multiple parameters of the treatment data are aggregated for multiple patients, along with outcomes data and expected likely outcomes of CAPD treatment are modelled as a function of said multiple parameters of treatment data, to provide to assist in prescription optimisation and patient centric treatment.

### FIELD OF THE INVENTION

This invention relates to continuous ambulatory peritoneal dialysis (CAPD).

### BACKGROUND TO THE INVENTION

Patients who suffer from End Stage Renal Disease (ESRD) can be treated for their kidney failure with different modalities including various forms of dialysis or by way of kidney transplantation. One of the modalities of dialysis is Peritoneal Dialysis (PD) which is available by way of two modalities, one being Automated PD (APD) and the other being Continuous Ambulatory Peritoneal Dialysis (CAPD).

CAPD is typically performed manually by a patient four times per day. Dialysis fluid/solution (dialysate) is introduced into the patient's peritoneal cavity by means of an in-dwelling peritoneal catheter and infused by gravitational flow with a bag with the dialysis solution being hung above the patient's abdomen. The dialysis solution dwells in the peritoneal cavity of the patient for about four to six hours. The peritoneal membrane acts as the diffusion barrier for exchange of excess water, metabolites and other waist products to be eliminated from the body. At the end of this time, fluid containing water and waste metabolites is drained from the patient's cavity, under the influence of gravity. Once more fresh dialysis fluid is infused into the cavity to continue the process.

The patient carries out the drain and fill cycle noted above by executing a predetermined sequence of steps to first drain spent fluid and then to refill the peritoneal cavity with fresh fluid. Performing each exchange requires a predetermined sequence of steps of opening and closing, several pre-connected tubes in a closed system of bags and tubes that has been defined as a Flush-Before-Fill Peritoneal Dialysis closed system. These are commonly now referred to as the “twin-bag” procedure or variations of such a procedure.

The patient is typically treated by a specially trained renal physician normally with a qualification as a Nephrologist, with the support of a PD Nurse who has specific training in the care of PD patients. Together they make up with other medical professional the healthcare practitioner team (HCP).

Known commercially available CAPD systems require many clamping and unclamping steps for a single exchange. For example, a typical system may require the following manual steps:
1. The patients need to conform to the protocol of their PD unit training manual for which they have undergone approximately two weeks of training and a competency assessment on completion before they can perform PD at home on their own.
2. The patients need to be trained on the various PD solution products that they will need over the course of their treatment which requires them to follow a prescription generated by their HCP supervising their treatment
3. The patients need to adjust their daily routine to accommodate the need to perform on average four PD exchanges per day;
4. The patients need to remember which specific PD solution they require at each PD exchange interval;
5. There are different manufacturers of PD dialysate solutions, and they use different technologies and some patients may be required to use different technologies during the course of the same day;
6. The last PD exchange of the day is invariably of longer duration until they perform their next PD Exchange the following day and this specific PD exchange invariably utilizes a special solution concentration and physical contents that are different from the other PD dialysate solutions, they would use the rest of the time;
7. The patients need to remember at the time of every PD exchange to perform a series of preparatory steps to ensure they have the correct product, the correct materials and other items for them to perform a safe exchange.
8. The major risk a patient needs to guard against is an episode of peritonitis, which is infection of the peritoneal membrane. The major cause for peritonitis is attributed to S. Aureus from touch contamination or inadequate hygiene practices.
9. The patients are also expected to meticulously record all the events of their PD exchange such as the solution they have used, the volumes that were drained and filled respectively and other onerous details. These recordings are captured in a standard book and written by hand.
10. The patients have a range of PD dialysate solution products that they can use and while they are expected to perform each exchange as per the prescription of their attending physician, they are also able to substitute products that may give them a different dialysis outcome;
11. Once the patient has performed the various preparatory steps and ensured that he/she is suitably disinfected, he/she would connect to the abdominal catheter connector;
12. The patient opens a specified clamp to drain the dwelling solution from his/her abdomen to an empty bag lying on the floor;
13. The patient needs to monitor the drain bag to ensure that it is indeed filling up with effluent dialysate and cannot estimate if the flow rate is slow, which would indicate a potential mechanical defect of their catheter and therefore needs to ensure that they the catheter has not been blocked or that flow is not impeded by the patient's physical position, mechanical failure of the catheter is considered, along with peritonitis, one of the major causes of technique failure for PD;
14. The patient closes the specified drain clamp after the drain cycle;
15. The patient places and closes a specified clamp on the tubing connecting the sterile solution bag containing fresh dialysate;
16. The patient breaks a frangible seal or other such device that seals the fresh solution bag within the tubing system;
17. The patient opens the upper clamp on the solution tube and the lower clamp on the drain tube to start a flush cycle;
18. The patient estimates approximately 150 ml of fluid that has left the solution bag and then closes the clamp on the solution tube once more and also the clamp on the drain tube to end the flush cycle;
19. A major risk for patient safety during this flush-before-fill procedure is that the catheter connector may not have been closed and fluid with possible contaminants in the tubing is infused into the patient's abdomen, thus being another major source of peritonitis contamination;
20. The patient opens the clamp of the solution tube to start infusing dialysate solution into the abdomen which is referred to as the fill;
21. The height of the PD dialysate solution is variable, which may well affect the flow rate of the solution;
22. The flow rate of the PD dialysate solution is purely governed by gravity and resistance from the Intra-Abdominal Pressure of the patient and this can cause considerably pain on infusion or other discomfort as the solution may be too cold, or flow too fast;
23. The patient needs to monitor their fill bag to ensure that it is emptying of fresh dialysate and cannot estimate if the flow rate is slow, which would indicate a potential mechanical defect of the catheter and the patient therefore needs to ensure that the catheter has not been blocked or that flow is not impeded by the patient's physical position, as also referred to in point 10;
24. The patient needs to estimate how much fluid has flowed under gravity into the abdomen and then stop the fill by closing the clamp on the solution tube;
25. This procedure is limited to the volume of solution in the bag during production and cannot be measured or controlled accurately, making this modality unsafe for patients of smaller anatomical size and thus lower Body Surface Area (BSA) or for children.
26. There is standard manufacturing procedure for PD dialysate bags to have volumes +10% of the volume shown on the solution bag, allowing for a range of 1,800 ml to 2,200 ml of Dialysate solution being infused by a patient, however the estimation of fluid volume is covered in point 30;
27. The patient closes the clamp on the solution line to end the fill once the solution bag is empty;
28. The patient needs to close the catheter connector and disconnect himself/herself from the CAPD bags;
29. The patient must control the drain bag containing the waste products and determine if there are any indications of foreign bodies associated with complications and possible early indication of infection;
30. The patient needs to record all the volumes that he/she estimates were drained and infused during that specific exchange;
31. The patient needs to calculate the Ultrafiltration (UF) of the specific exchange which is calculated as:
Drain Volume−Previous Fill Volume
   and determine if the UF is sufficient, according the preceding UF measurements already achieved in previous exchanges, as well as according to a UF target that has been provided to the patient from the attending HCP;
32. The patient needs to notify the PD unit nurse if the drain bag has any contaminants referred to in point 13;
33. The patient needs to take a recording book with the data recorded along with him/her on his/her next appointment at the PD unit;
34. The products that have been used to achieve the UF results referred to in point
31 may well not be the products that have been used for the specific PD exchange as explained in point 5;
35. The attending physician, or other qualified healthcare practitioner, are required to provide a prescription for the CAPD patients according to standard of care guidelines established by international bodies and on evaluation of the clinical features of the patient. There are currently no CAPD prescribing tools available to them to allow for decision support related to the most appropriate prescription for the patient.
36. The attending physician, or other qualified healthcare practitioner, are only able to provide a prescription for the CAPD patients which is today limited to a few variables, such as the type of solution in a volume that is commercially available and the frequency per day with the possibility to prescribe solutions that may or may not have better treatment outcomes.
37. The fill volume of the dialysate that is prescribed is calculated on a premise that a patient's weight and height and therefore their body mass index are the criteria by which the fill volume for the prescription is decided.
38. The attending healthcare practitioners (HCPs) are unaware of the progress of the patient's treatment, save for the routine clinic visit appointment that the patient may attend or informal telephone conversation with the HCP staff and not be able to be informed of potential side-effects.
39. The measurement of the accumulated daily treatment outcome is not able to be measured accurately by the healthcare practitioners and they are also not able to receive the information with any frequency.
40. There are no early notification indices available to the healthcare practitioners to allow them to monitor their patients while they are performing CAPD in their homes.
41. The decline of UF, or indeed other clinical parameters such as temperature, over a few days has been shown to be an indicator of peritonitis however for an attending physician it is not possible to measure these changes for their CAPD patients
42. HCP staff at hospitals not directly caring for the ESRD patient may not have any indication of how to perform the PD exchange if the patient is hospitalized under their care
43. The PD unit entrusted with the care of the patient may not have attendant staff 24 hours per day and therefore not be able to attend to any emergencies the patient may experience.

Oreopolous first described the Continuous Ambulatory Peritoneal Dialysis procedure in the early 1980's. Since that time there has been limited innovation for CAPD patients around the world. There was the development of the dual-chamber bag approach with so called Y-set connection to the patient and the embedded Flush-before-fill procedure to minimise the number of activities needed to perform the dialysis, which had an improvement in the peritonitis rates experienced by patients. Peritonitis is one of the leading causes of technique failure. The Tenckoff catheter that is used has remained largely unchanged in all the time of the CAPD treatment and the failure of the catheter is classified as Mechanical Complication, which is invariably another major cause of technique failure as referred to in points 10 and 23 in the above list of steps followed by patients.

Innovations to reduce touch contamination which are also causes of peritonitis have seen various innovations like the use of luer-taper type connection systems or other such connection systems attempting to reduce mistakes during critical moments in the performance of dialysis exchanges. There has been the introduction of different dialysate solutions to attempt to reduce the exposure of glucose degradation products on the peritoneal membrane and developments in the types of solutions continue. Lastly the other main area of innovation has been an attempt to alleviate the patient needing to manually clamp the tubing in critical phases of the dialysis procedure with the introduction of so-called dialer technologies to assist the patient. None of these innovations have been a distinct reason for improving CAPD outcomes on their own.

It must also be pointed out that a lot of innovation and investment has been made in a modality similar to CAPD but different and that is automated peritoneal dialysis APD.

This Procedure uses similar dialysate solutions, the same Tenckoff catheter to access the peritoneal cavity and similar luer taper or such like connection technologies to the Tenckoff catheters but otherwise is a distinctly different form of peritoneal dialysis.

Therefore the performance of CAPD is still predominantly a manual process, conducted by the patient manually and in accordance to a training programme provided at the initiation of treatment and reliant on the patient to record the limited amount of information about the treatment in a written notebook. This information being largely an estimation and an indication of how often the patient performs their treatment and how compliant they are to the procedure.

Fabian Eibensteiner et al, “Monitoring Daily Ultrafiltration in Automated Peritoneal Dialysis” (CJASN 17:1-4, 2022) further articulated the critical issue as: “Efficacy of peritoneal dialysis (PD) is often time-limited by accompanying chronic damage to the peritoneal membrane. High-quality, goal-directed dialysis is characterized by adequacy of peritoneal ultrafiltration (UF) and small solute clearance while maintaining fluid and salt homeostasis. Occasional measurements of clinical parameters of peritoneal membrane dysfunction in individual PD patients are highly variable. Inspired by time-series analyses such as Holter electrocardiograme studied automated PD (APD) cycler readouts on daily inflows and outflows of PD fluid dwells to investigate the value of continuous UF monitoring under clinically relevant conditions. Daily peritoneal UF monitoring by continuous APD data differs from traditional clinical data in quantity, noise, and missing normative values. Such analysis must consider unique patient trajectories, intra-individual variability, data noise and temporal autocorrelation. Our study demonstrated that continuously collected data allow analysis of unexpected, otherwise undetected PD-related events and their effects on UF. Daily UF changes are correlated with evolving peritoneal membrane function during long-term PD, in relation to clinical risk factors and outcomes. Increased use of APD cyclers with remote patient monitoring will generate large datasets for real-time treatment analysis through cloud-based services, allowing timely treatment modifications to improve patient outcomes. Therefore, continuous UF measurement with automated analysis could monitor peritoneal membrane function and alert clinical teams upon detection of relevant changes, without need for serial ”screening“ membrane tests.”

As there are no CAPD devices available on the market today that compare to the available APD devices it may be argued that there is a desperate need for such a device for various reasons.

CAPD is still considered to be an effective form of renal replacement therapy (RRT) for patients suffering from advanced stage renal disease (ASRD)
CAPD may be considered a better modality of treatment than APD with numerous advantages but most especially the preservation of residual renal function (RRF)
A brief summary of the benefits of CAPD compared to APD may be: concerns regarding increased cost, a more rapid decline in residual renal function, inadequate sodium removal and disturbed sleep are APD's setbacks. Besides APD superiority over CAPD in fast transporters, any other medical advantages of APD still remain controversial
It must however be acknowledged that the major challenge of CAPD is technique survival for several reasons. Some of these relate to the inability to manage the patient, as would be expected given the lack of accurate anthropometric measurements.

The proportion of patients performing continuous ambulatory peritoneal dialysis (“CAPD”) is increasing worldwide, which is due in part to CAPD providing good clinical outcomes for a patient and allowing the patient to lead an economically active life. The two primary goals of dialysis, solute clearance and ultrafiltration (“UF”) depend on a number of variables and also the compliance of the patient to perform their prescription at a particular time. However, these may also be influenced by a number of specific variables such as the PD solution type, therapy duration time and fill volume. However there may also be additional factors such as etiology of the patients renal failure, the presence of additional co-morbidity diseases the patient may be afflicted with, the clinical presentation of the patient, the additional medication the patient may already be prescribed to manage the existing diseases. Prescribing a CAPD therapy constitutes selecting one of each of these parameters, as well as the patient needing monitor themselves for important signs of well-being and potential side-effects of the treatment. There are many combinations and possibilities from which to choose, and the patient is trained during their enrolment and competency assessment to perform CAPD, to remember each step of the procedure they need to perform manually. It must also be remembered that because of the nature of their disease some patients may well suffer cognitive decline and therefore find the task of performing their treatment safely a challenge.

CAPD treatment typically relies on the patient to perform their treatment safely and then capture all their treatment data manually or, as in the recent past, capture it manually in a digital format. The patient is also expected to manually estimate the volumes that they have drained, the amount of solution they have flushed in the flush before fill procedure and the volume they have filled in their abdomen. No important data like the flow of the solution or the actual time that the various sections of the treatment take are possible to be measured. The patient is also expected to remember their prescription and use a specific product they have been prescribed and also trained to use in specific circumstances. However patients may be expected to alter the solutions they use but have based their calculations on the assumption of volumes and their manually recorded UF for a specific treatment.

A patient is also expected to perform four CAPD dialysis exchanges a day and the healthcare practitioner is unaware of how many dialysis treatments the patient has actually performed. It is also typical for most patients that after a few months of treatment have lapsed, they start to neglect to capture all their data they should record for each treatment. The patient does not have any tools to easily communicate their treatment results to their healthcare practitioners. Accordingly, some patients underachieve their targets and develop adverse conditions such as fluid overload and in some cases hypertension. Additionally, as mentioned above, when there are indications that patients are starting to experience side-effects of their dialysis treatment, they may be inclined to ‘wait a day’ before notifying their healthcare professional and this may result in potentially harmful medical conditions.

A CAPD patient may also be denied the ability to have an automated peritoneal dialysis (APD) machine, as these require electricity, and the patient may well not have reliable electricity supply. Alternatively, they may not be able to afford the cost of the much more expensive dialysis option of APD. These methods place the burden of therapy solely on the patient. The healthcare practitioner has to devise ways to attempt to stay abreast of the patient outcomes of treatment to assist the patient. Otherwise, they are only left with the ability to assess a patient once per month at a clinic appointment, which may sometimes even be longer than four-week intervals.

Provision of renal replacement therapy and specifically home-based Peritoneal Dialysis is a challenging and complex treatment for healthcare professionals to manage. Innovative medical device systems are allowing for the management of PD patients to be improved. However, new medical device regulations are also requiring a higher degree of oversight, particularly in respect to the clinical evaluation of the performance of these medical device systems. Data that is routinely generated lies at the basis of the ability to demonstrate the performance specifically with medical device software, where real world experience needs to be actively managed. The importance though to maintain compliance to ethical standards is critically important and working mechanisms should be articulated prior to engagement.

Guyton's model of how the kidney works is the centre-point of long-term blood pressure control and described as the “pressure natriuresis relationship.” A relationship between renal perfusion pressure, sodium excretion and blood pressure as an important mechanism for regulating circulatory volume. The kidneys regulate circulatory volume by controlling sodium and water balance, thus maintaining extracellular fluid volume (ECFV) homeostasis. End Stage Renal Disease (ESRD) and Renal Replacement Therapy is thereby a challenging disease to manage.

Ultrafiltration (UF) has been shown to be the one parameter that is directly correlated with mortality in CAPD patients and ultimately has led to the concept of “Prescribing High-Quality Goal-Directed Peritoneal Dialysis”.

Continuous Ambulatory Peritoneal Dialysis (CAPD) as a comparator to other dialysis modalities has been shown to have beneficial effects on residual renal function.

It is well documented that CAPD has two main burdens: The burden of treatment experienced by the healthcare providers, and the burden of therapy experienced by the patients. However treatment technique survival is also known to have several measurable quantifiable and qualitative preventative risk factors that can impact the technique survival. Clinically most notably infections, leakage and catheter problems are important modifiable causes for technique failure.
Direct procedural mistakes may have an immediate impact, as an example, potential exposure to contamination that needs to have immediate response by initiating treatment and monitoring of the patient.
Early identification and notification of contaminated PD drained effluent may prevent hospitalisation and even improve technique survival.
Non-compliance to one treatment prescription is not significant but trend analysts identify patient burden.
Little procedural mistakes which don't have direct impact on the PD exchange, over time indicate the patient is starting to demonstrate symptoms of burn-out.

Remote patient care monitoring of PD patients is highlighting the re-organisation of PD medical teams allowing greater proactive management of patients and thereby reducing the burden of treatment.

Treating ESRD patients is complex as each patient has different disease states (causes) and aetiology and demographics. The effect of medication prescribed for the patient to treat either the direct cause of their renal failure or additional medications prescribed on the outcome of the patients dialysis outcomes is not able to be measured by the attending physician. More specifically on a daily basis and the establishment of trends over time to understand a specific patient's response treatment. Currently there are fixed models of prediction based largely on retrospective analysis and are limited to only a few parameters that are able to be considered. How an individual patient responds is also not understood as the attending physicians can only review the actual clinical status of the patient infrequently.

The combination therefore of the condition of the patient, their response to the specific CAPD prescription, their compliance to the CAPD prescription as well as the impact the additional treatment that is provided to the patient for various other diseases is only able to be reviewed on an infrequent basis. Specific response to certain PD solutions, the volumes that are used and the impact the combination of the chemical and physical properties of the PD solutions have on the outcomes is not able to be measured. The dynamic response of the patient's body to the filling and draining of the PD solutions is also not able to be measured and while it is known these kinetic forces are present and their impact may be felt, it is today not possible to measure these. The attending physicians need to rely on static measurements to understand dynamic forces to try and provide the best care for their patients.
The fill volume for a prescription has an existing formula however the forces that affect the Intraperitoneal pressure (IPP) are not known
The infusion is known to affect the IPP and IPP changes during PD exchange, but it is currently only monitored by static measurements, which could obscure true (and more importantly, elevated) IPP levels that pose danger to the patient.
It is know that IPP measurements that exceed certain levels may directly cause herniation and also may affect heart function, but elevated IPP could go undetected, or not come to the attention of the HCP, if it is only measured statically and infrequently.

Medical device regulations (MDR), e.g. in the EU, require that the manufacturer create clinical evaluation plan that demonstrates the claims of the medical device are founded and reviewed over time.
The medical team not only need to manage their individual patient but also manage the PD programme to international standards. Data analysis is at the heart of both requirements.
The essential rights of the patient and informed consent need to be navigated with the need to use data, with due care for safety and anonymity, in order to maintain treatment efficiently and remain within legislative requirements.
However, a clinical evaluation plan (CEP) and subsequent frequent reporting is also needed to ensure that the medical device is performing as claimed and also in compliance to local competent authority regulations. Legacy medical devices already placed in the market of the EU, do not require clinical investigation, but nevertheless need to comply with the new MDR-CEP requirements and similar requirements exist in other jurisdictions. One of the recommendations is the supervision of the CEP and is performed on behalf of the manufacturer by a suitably qualified expert (medical advisor) supported by additional experts in the field.
Local institutions also have adopted procedures to evaluate a new medical device prior to accepting the device as a standard medical device to be used within their procurement procedures. The evaluation requires controlled use by patients within a limited time and following the internal protocols as established and adopted. As an example 45 patients may be enrolled with performance and usability variables monitored and analysed over a set period. Thereafter the review would recommend the use of the medical device or decline continuing.

In this specification, reference is only made to some errors, but the present invention concerns two major categories of errors: clinical errors and Technical errors. Clinical errors can be divided between: critical, potentially life-threatening errors; and non-critical errors. Technical errors can similarly be divided between critical and non-critical errors. It is essential that critical errors be identified and actioned as soon as possible.

The present invention attempts to remedy or ameliorate the abovementioned shortfalls of the prior art.

### SUMMARY OF THE INVENTION

According to a first aspect of the present invention there is provided apparatus for performing continuous ambulatory peritoneal dialysis (CAPD) for a patient, said apparatus comprising:
programmable circuitry on which a healthcare provider can enter prescription data;
a user interface that is configured for the patient to enter data and to provide information to the patient, said information including at least sequential instructions to the patient to perform the CAPD treatment according to the prescription data;
instruments for gathering treatment data including at least one of: dates and times when CAPD treatments are preformed; durations of flushing, filling and/or draining; quantities of dialysate solution used during flushing, filling and/or draining; and intraperitoneal hydrostatic pressure (IPP);
a communication module configured to communicate the treatment data to the healthcare provider.

The instruments for gathering treatment data may include at least one weigh scale that is configured to weigh a dialysate solution container, said weigh scale may include an analogue load cell and circuitry configured to convert an analogue signal emitted from the load cell to a digital signal.

The circuitry of the weigh scale may be configured to convert variations in weight of the dialysate solution container to the quantity of dialysate solution used during flushing, filling and/or draining, using the specific gravity of the dialysate solution, and may be configured to convert variations in weight of the dialysate solution container over time to determine a flow rate of the dialysate solution. The programmable circuitry may further be configured to convert the flow rate of the dialysate solution to IPP.

The communication module may be remote from the instruments for gathering treatment data, and the apparatus may include circuitry configured to time/date stamp the treatment data.

The user interface may be configured to obtain feedback that the CAPD treatment is being administered correctly.

The user interface may be configured to log mistakes made by the patient, to provide an indication of the patient's ability to cope with the burden of treatment and to alert the healthcare practitioners if a serious mistake has been committed.

The instruments for gathering treatment data may include at least one flow management constrictor that is configured to measure the type of dialysate solution and concentration of said dialysate solution, said flow management constrictor containing photometric instruments and circuitry configured to convert chemical and physical properties of the dialysate solution to digital information. The circuitry of the flow management constrictor may be configured to measure variations in the nature of a plastic type tube in which the dialysate solution flows, to verify properties of material in the tube. The programmable circuitry of the flow management constrictor may be configured to verify that the nature of a solution that is being infused accords with the feedback provided the patient on the user interface.

According to another aspect of the invention there is provided a system for performing comprehensive management of continuous ambulatory peritoneal dialysis (CAPD) for a patient, said system comprising apparatus as described herein above, wherein the healthcare provider is remote from the apparatus for performing CAPD and the communication module is configured to communicate the treatment data to the healthcare provider, remotely.

The communication module may be configured to allow the healthcare provider to enter prescription data remotely on the programmable circuitry, may be configured to communicate the treatment data to the healthcare provider in real time, and/or may be configured to allow the healthcare provider to enter prescription data remotely on the programmable circuitry in real time.

According to a further aspect of the present invention there is provided a method of managing continuous ambulatory peritoneal dialysis (CAPD) of a person in need thereof, said method comprising:
entering prescription data on programmable circuitry;
understanding potential outcome of the use of the programmable circuitry communicated prescription by using aggregated data of individual CAPD treatment history or of information gathered and interpreted to affect such expectation;
generating interactive information and sequential instructions in the programmable circuitry, said interactive information and sequential instructions being for the performance of CAPD treatment according to the prescription data;
displaying the interactive information and sequential instructions on a user interface;
gathering treatment data including at least one of: dates and times when CAPD treatments are preformed; durations of flushing, filling and/or draining; quantities of a dialysate solution used during flushing, filling and/or draining; potential hazardous events that may have occurred during the treatment; and intraperitoneal hydrostatic pressure (IPP); and
communicating the treatment data to a healthcare provider via a communication module.

The method may include aggregating the treatment data for the benefit of the healthcare provider and communicating such aggregated data in manners that are effective and efficient for the healthcare provider, and may include disseminating information gathered of the treatment data and related information to appropriate role-players so configured to receive and respond to such data.

The step of gathering treatment data may include weighing at least one dialysate solution container on a weigh scale. The weight scale may include an analogue load cell and the method may include converting an analogue signal emitted from the load cell to a digital signal.

The method may include converting variations in the weight of the dialysate solution container to quantity of the dialysate solution, using the specific gravity of the dialysate solution, and may include converting variations in the weight of the dialysate solution container over time to a flow rate of the dialysate solution. The method may further include converting the flow rate of the dialysate solution to IPP.

The communication module may be remote from locations where the CAPD treatments are performed and the treatment data are gathered, and the method may include time/date stamping the treatment data.

The method may include obtaining feedback from the person in need of CAPD treatment via the user interface, that the CAPD treatments being administered correctly.

The method may include logging mistakes made by the person in need of CAPD treatment and generating an assessment of the person in need of CAPD treatment's ability to cope with a burden of treatment or if indeed they require urgent medical attention.

The method may include measuring a type and a concentration of the dialysate solution by converting chemical and physical properties of the dialysate solution to digital information using photometric instruments and circuitry on at least one flow management constrictor. The method may include measuring variations in the nature of a plastic type tube in which the dialysate solution flows, to verify properties of material in the tube and/or the method may include verifying that the nature of a solution accords with the feedback provided by the person in need of CAPD treatment on the user interface.

The method may include measuring and calculating the flow rates of dialysate as well as the positive and counter-acting forces that may impact the flow rates. Results of the measuring and calculating may result in the determination of dynamic measurement of safe levels of pressure within which the treatment is considered efficient. Such measurements may also provide the decision making of future prescriptions in order to ensure patient safety and comfort.

The method may further include:
aggregating the treatment data gathered for multiple said persons in need of CAPD, said treatment data including multiple parameters;
gathering and aggregating outcomes data for said persons; and
modelling expected likely outcomes of CAPD treatment as a function of said multiple parameters of treatment data, by reviewing said treatment data and outcomes data.

The method may include determining Peritoneal Equilibration Test (PET) statuses for each of the persons in need of CAPD and evaluating efficacy of PET against expected outcomes of CAPD.

The method may include aggregating blood pressure data from said multiple persons in need of CAPD and modelling blood pressure expected as a function of said multiple parameters, and/or determining an optimised prescription for a person in need to CAPD.

The method may include determining the dynamic specific intraperitoneal pressure IPP of a person in need of CAPD, which may be used as a single measurement for the prevention of a side-effect or used to evaluate the efficacy of a CAPD treatment outcome.

The method may include assessing a combination of measurements that in isolation may not provide interpretation of meaning however when considered together may provide invaluable information directly impacting response to the information.

The method may further include:
collection and availability of relevant data required to create prescriptions and allocate specific apparati to specific patients;
provision of adjunct data provided to facilitate supplementary information and further describes the patients' condition or activities ; and
advancing whole person healthcare with the emphasis on aggregating data for multi-factor analysis.

### BRIEF DESCRIPTION OF THE DRAWINGS

For a better understanding of the present invention, and to show how it may be carried into effect, the invention will now be described by way of non-limiting example, with reference to the accompanying drawings in which:

FIG. 1 shows a three-dimensional view of an embodiment of a CAPD device according to the present invention;

FIG. 2 shows a diagrammatic representation of the CAPD device of FIG. 1, in use;

FIG. 3 shows a screenshot of a dashboard displayed by medical device software (MDSW) on a user interface of the CAPD device of FIG. 1;

FIG. 4 shows an example of a physician dashboard; and

FIG. 5 shows a diagrammatic representation of an embodiment of a system according to the present invention.

### DETAIL DESCRIPTION OF THE DRAWINGS

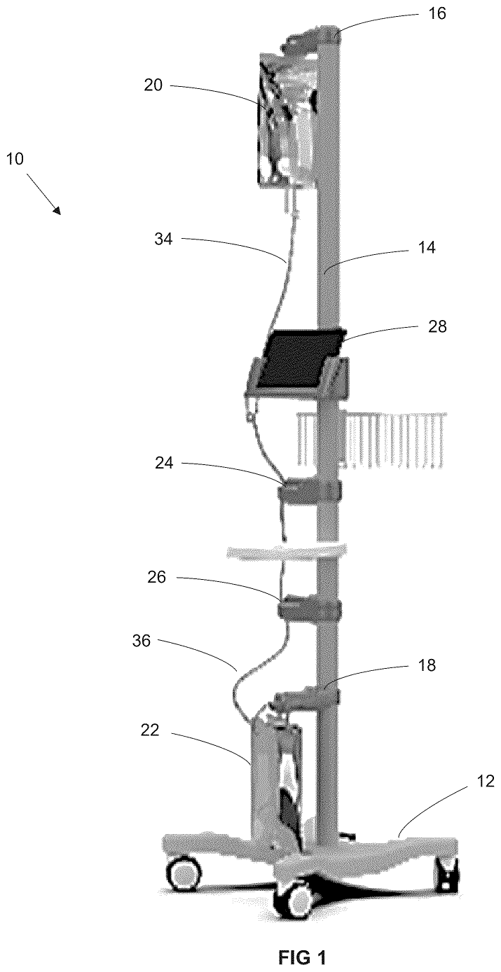

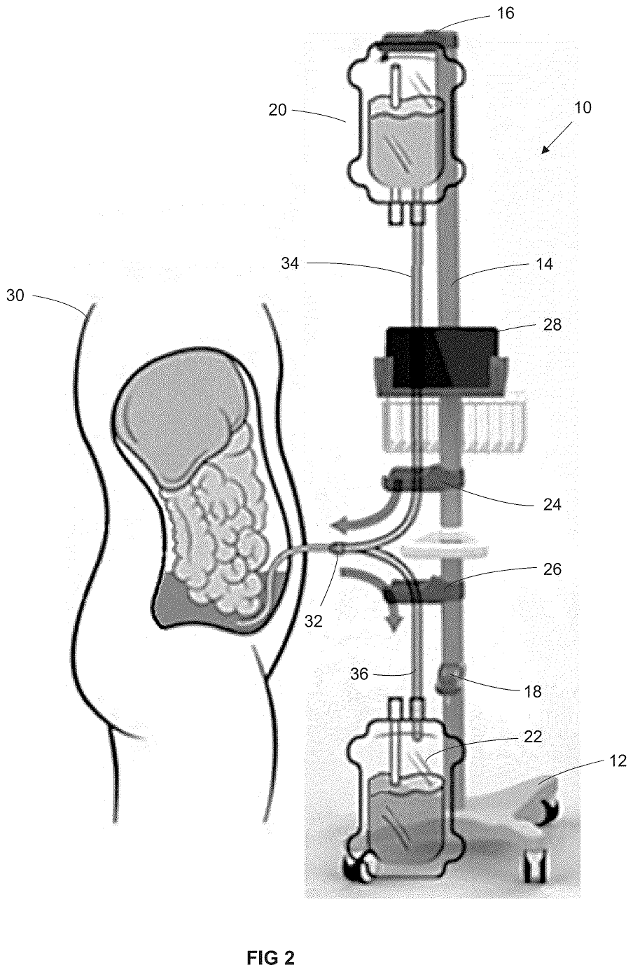

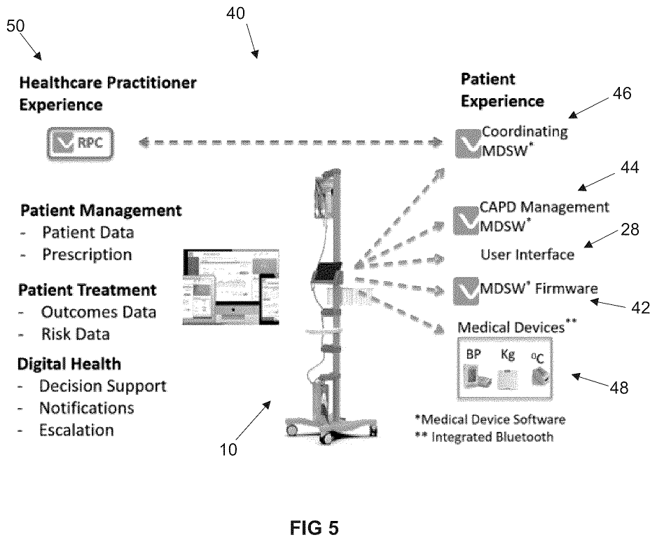

The present invention uses a system for performing CAPD on a patient and a preferred embodiment of the system includes a CAPD device as shown in FIGS. 1 and 2 and identified generally by reference number 10. The system is shown in FIG. 5 and is identified generally by reference number 40.

In the illustrated embodiment, the CAPD device 10 includes a mobile base 12 and an upright post 14 for supporting parts of the device at preferred elevations, but in other embodiments, the device could include a different support structure.

The CAPD device 10 includes an upper weight scale 16 that is supported at the top of the post 14 at a height of 174 cm and a lower weight scale 18 supported on the post 14 at a height of 37 cm. The heights at which the weight scales 16, 18 are supported, like heights of other parts of the CAPD device 10 that will be described below, are measured from a floor on which the CAPD device is supported, and are merely non-limiting examples of suitable heights-that can be varied in other embodiments of the invention. However, it is important for aspects of the invention that will be described below, that the heights of the weight scales 16, 18 are known.

In some embodiments of the invention, the CAPD device 10 could include only a single weight scale, preferably the upper weight scale 16, but two weight scales is preferred.

Each of the weight scales 16, 18 includes a cantilever arm with an attachment formation such as a hook that is configured to support a bag filled with dialysate solution. In use, a bag 20 for supplying fresh dialysate solution will be supported by the upper weight scale 16 and will be referred to herein as the “top bag 20” for brevity. Similarly, a bag 22 for receiving fluid drained from a patient's peritoneal cavity (i.e. effluent or spent dialysate) will be supported by the lower weight scale 18 and will be referred to herein as the “bottom bag 22”.

Each of the weight-scales 16, 18 is configured to weigh the bag 20, 22 it supports, preferably with high accuracy, e.g. by using load cell technology. The upper weigh scale 16 monitors the weight of the top bag 20 to monitor dialysate infusion into a patient's peritoneal cavity and the lower weight scale 18 monitors the weight of the bottom bag 22 to monitor spent dialysate drained from the peritoneal cavity under gravity. Both weight scales 16, 18 monitor these weights over time and record data.

The CAPD device 10 includes an upper clamp 24 at a height of 77 cm and a lower clamp 26 at a height of 45 cm and each of the clamps is supported by the post 14. Each of the clamps 24, 26 is configured to clamp a flexible tube and to serve as a flow management constrictor. In some embodiments of the invention, the CAPD device 10 could include only a single clamp, preferably the upper clamp 24, but two clamps is preferred.

Each of the clamps 24, 26 includes a constriction mechanism that is able to recognise the physical properties of tubing received in jaws of the clamp and that is configured to clamp the tube to control the flow rate of fluid in the clamp. Each constriction mechanism is automated and can constrict the tube to stop or prevent fluid flow in the tube, or to restrict and control the flow rate of fluid in the tube.

In a preferred embodiment, each clamp 24, 26 is also equipped with an optical sensor that is configured to monitor fluid flowing in a tube held by the clamp. The optical monitoring can be used to monitor colour of the fluid, inclusions in the fluid, opacity of the fluid, or the like. In particular, each clamp 24, 26 preferably includes photometric instruments and circuitry configured to convert chemical and physical properties of the dialysate solution to digital information.

The CAPD system 10 also includes a user experience device or user interface, which in the illustrated example, takes the form of a tablet computer 28 that is supported by the post 14. The tablet 28 is for interaction with the patient during the CAPD procedure and displays output that guides the patient through the procedure, but the tablet 28 also performs tests and monitor conditions of equipment to ensure procedural compliance. The tablet 28 and its associated processing circuitry also captures data including the times when CAPD is performed, the duration of different steps of the process, such as flushing, filling and draining.

The Tablet 28 provides the processing capacity for the CAPD device 10 and various proprietary medical device software/s (MDSW) on the tablet receives input from the weight scales 16, 18 and the clamps 24, 26 and it controls the clamps. In the illustrated embodiment of the invention, there are multiple MDSW programs in the form of application software or “apps” that operate on the tablet. The MDSW apps running on the tablet 28 include firmware 44, an app 44 for CAPD management, which only generates CAPD data, and an app 46 for coordinating the patient's prescription and capture non-CAPD data such as blood pressure, body weight and temperature. In other embodiments of the invention, there may be more than one device with processing capacity, which may form part of the user interface, there may be fewer or more apps, which can perform the same functions as the apps running on the tablet 28 in the illustrated example. However, the presently preferred embodiment of the invention uses firmware and two apps on a single tablet 28, that provides all the processing capacity of the device 10,

The system 40 can also monitor other parameters such as pressures and flow rates of dialysate, anthropometric data such as weight, blood pressure, etc. The system preferably includes sensors 48 that can communicate with the tablet 28, such as a thermometer, scale and blood pressure monitor that can communicate wirelessly with the tablet. e.g. by Bluetooth. Patients operate the thermometer, scale and blood pressure monitor manually, but data measured by the devices are automatically transmitted to the tablet 28, where it is captured and processed.

The data captured in the CAPD system 10 is preferably stored and is communicated, or made available to either the patient directly or a healthcare practitioner. The data can be processed, or processed in part in the tablet 28 and can be stored on the tablet, from where it can be accessed on demand, or the data can be transmitted by wireless communication, e.g. via the internet, can be stored in cloud-based storage, can be transmitted and stored on a remote server, or the like.

Referring to FIG. 2, when a patient 30 uses the CAPD device 10 to perform CAPD, the patient attaches the top bag 20 to the upper weight scale 16 and the bottom bag 22 to the lower weight scale 18 and attaches the tubing from the bags to the patient's catheter 32 in the conventional manner. The upper tube 34 leading from the top bag 20 is inserted between the jaws of the upper clamp 24 and the lower tube 36 leading to the bottom bag 22 is inserted between the jaws of the lower clamp 26.

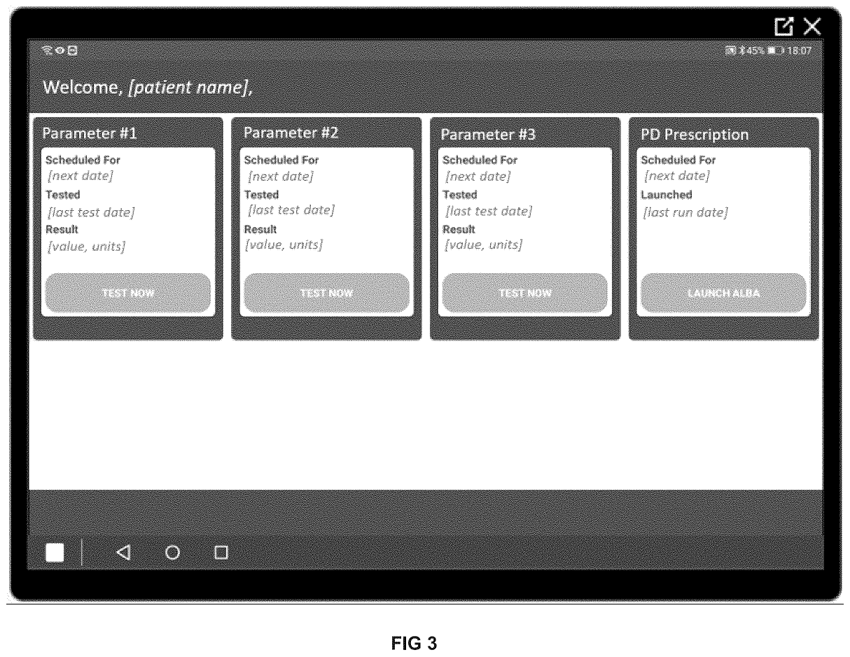

The patent is prompted during the preparation and conduct of the CAPD exchange and afterwards, with instructions issued by the CAPD management app 44 and displayed on the screen of the tablet 28. Some of these instructions require confirmation by the patient that procedural steps have been completed and some prompts require that the patent enter information or make menu selections. The patient is thus engaged during the CAPD exchange and is guided through the process, while data relating to the CAPD exchange are continuously captured. An example of a dashboard displayed to a patient on the tablet 28, is shown in FIG. 3.

The coordinating MDSW app 46 includes coordinator logic able to handle the complexity of CAPD therapy prescriptions by which to order and monitor a minimum of a clamp and/or weigh scale, provide comprehensive applicable information of each therapy prescription legally allocated by a healthcare practitioner duly authorised for a specific patient. In the illustrated embodiment, the coordinator logic forms part of the coordinating MDSW app 46 and runs on the processor of the tablet 28, but in other embodiments of the invention, the coordinator logic can instead or in addition run on a different electronic device, processor, system or network, that can communicate with the tablet. For example, the coordinator logic can run in a cloud-based logic coordinator.

The coordinating MDSW app 46 receive prescription data from a device 50 used by an authorised healthcare practitioner (or user). The prescription data is communicated to the CAPD management app 44 and is used to generate instructions/prompts to the patient, that are displayed on the screen of the tablet 28. Data gathered by the CAPD management app 44 on the tablet 28 during CAPD is communicated to the coordinating MDSW app 46, where it is processed and stored and from where it is made available to the user. The coordinating MDSW app 46 operates to accept or deny particular therapy allocated to the patient.

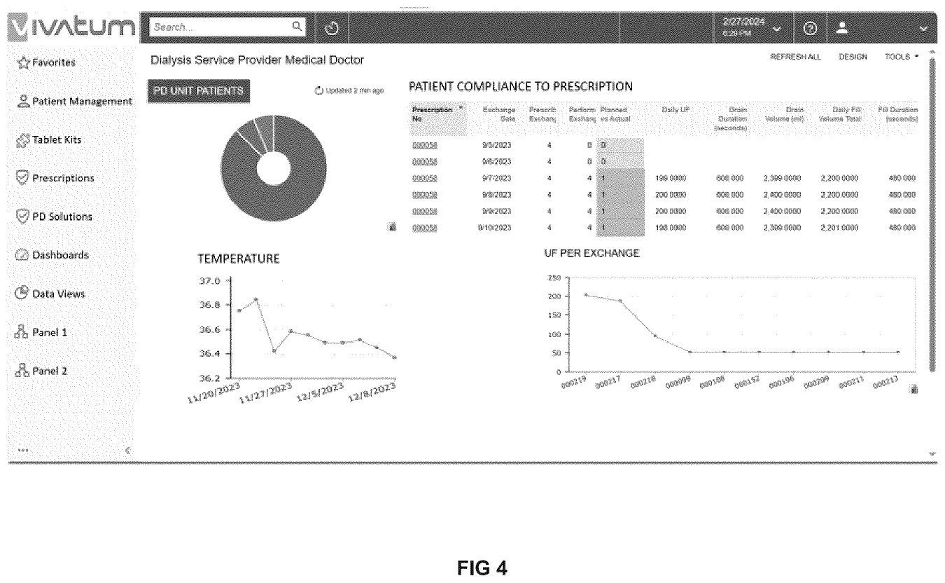

Another important component of the CAPD system is that the access structure of the logic coordinator allows a healthcare practitioner to choose an array of therapy prescriptions to be run on the CAPD device 10. A further component is that it is possible for the healthcare practitioner to receive a wide range of applicable information regarding how the patient has performed the various therapy treatments that were prescribed not only but including the results of the CAPD procedure. An example of feedback to a healthcare practitioner in the form of a physician dashboard, is shown in FIG. 4. Another component is that the patient is empowered to know his/her relevant treatment information and has the ability to be provided with historical results as to the outcomes that are measured during the performance of the treatment. An additional component, based on the programming of the logic coordinator is to suggest one of the therapy prescriptions to be run on the CAPD device 10 and courses of action one may be suggested to take.

A feature of the present invention is that a patient needs to adjust their daily routine to perform their dialysis, however each patient is unique and therefore should be able to arrange their required dialysis sessions to accommodate their needs. The patient and the healthcare provider can therefore organise together the most appropriate times the patient should try and perform their treatment. The default time arrangements should also be in a window of time and not a strict time event. The ability for a patient to know their daily routine but also then to be discreetly measured when indeed actual dialysis treatments are performed, help the medical team to assess how the patient is coping and address potential causes of burden of therapy the patient may be experiencing. Understanding the pressures that the patient may well be experiencing also allows the medical team to maybe adjust the prescription or to establish support structures to help the patient cope.

A further feature of the invention is the ability to understand the actual volumes that are infused into the patient's peritoneal cavity and how much is drained out of the cavity. This accurate information is up until today not known for CAPD patients. The time it actually takes for fresh dialysate to infuse into the peritoneal cavity and the time it actually takes to drain out of the peritoneal cavity is today not known for each patient. There is an assumption that the time is an approximation. Therefore the actual dynamic of how a CAPD patient's peritoneal cavity reacts to the treatment is also unknown. Pain on infusion of fresh dialysate, or pain on drain of effluent dialysate, is apparently experienced by several patients and there is no way to limit the flow of dialysate for a CAPD patient into their peritoneal cavity. Prior to the present invention, a patient who has a smaller body surface area, which then is directly proportional to the volume their peritoneal cavity can comfortably tolerate, needed to guess what volume they should infuse as generally there is only a 2 Litre dialysate solution bag available. The patient in question may also then be inclined to fill less than is prescribed and there is no real way of knowing this.

Another feature of the present invention allows for prescription improvement and enhanced customisation of the prescription for a specific patient. An important base line calculation of a patient is the peritoneal equilibration test or (“PET”). The PET determines amongst other features the characterisation of the patient's transporter type of their peritoneal membrane. The data collected for UF according to the present invention is much more accurate than the data samples used in prior art tests for CAPD patients. This directly assists developing a more accurate UF patient-specific characteristic curve. The data generated can then be utilised along with laboratory analysis to determine more accurate results. Knowledge generated will assist in helping the healthcare practitioners better suit the treatment for their individual patient.

A further feature of the present invention is the ability to monitor the prescriptions that the patients routinely perform along with the performance of the patient during their dialysis. The device 10 logs a range of performance characteristics while the patient interacts with the tablet 28. When a patient for instance attempts to perform a task that is not appropriate the MDSW running on the tablet 28 will recognise these tendencies by way of a performance log. The MDSW running on the tablet 28 has built in safety precautions and one such patient safety feature is to conduct a pressure test just prior to the Flush-Before-Fill sequence. If the patient has not closed their catheter connection this will be detected and is an indication that the patient has infused potentially contaminated solution into their abdomen. The tablet 28 will communicate this event urgently to the healthcare practitioners in order for them to initiate preventative treatment as soon as possible. Additional safety precautions include that in the even the tablet 28 is not connected to the internet the patient will be requested to immediately contact their healthcare practitioner and confirm they take responsibility. The prescription will also be adjusted to include temperature monitoring by the patient in order to assess the possibility of infection. In the event that the patient as similarly in another function such as at the time of the fill neglected to open their catheter connection, this will be recorded by the user interface. As there is no immediate health risk to the patient, the patient will be politely asked to close their catheter and this event will be recorded. If such an event occurs multiple times the healthcare practitioners will be able to initiate route cause analysis to assist the patient to perform the dialysis treatment safely by addressing needs that are elicited from the root cause analysis. The device 10, its integrated additional devices and other such forms of data gathering such as questionnaires and its interactive software can track several clinical outcome measures such as UF removed, body weight, blood pressure or also procedural events that can then cumulatively allow a better understanding of how the patient is performing.

A further feature of the invention is that flow rates of dialysate from the top bag 20 into the abdomen, and from the abdomen into the bottom bag 22 are determined accurately and continuously by the changes of the bags weight, detected in the weight scales 16, 18. The IPP for the patient is calculated in the coordinating MDSW app 46 from each of these flow rates, in an algorithm which uses the Bernoulli inter-relationship between the flow rates, the known elevations of the bags 20, 22, gravity, and the known density of the dialysates. The system 40 is thus capable of monitoring IPP continuously and can communicate IPP to the HCP 50. IPP is known to vary during PD, so measuring IPP only occasionally does not provide the HCP with sufficiently accurate information to detect potential risks from elevated IPP, such as heart failure or hernia.

A further feature of the invention controls how the information of differing prescriptions performs in respect to the outcomes achieved and other parameters and how these can be adjusted. Trends are very important for the healthcare practitioner to gauge how the patient outcomes are being achieved over time and therefore averages such as a weekly moving average for UF or any such useful trend analyses can be performed. The information available to the healthcare practitioner can also be customised to allow them to adjust their dashboards to accommodate their needs. Likewise a patient is, owing to the present invention, also able to work with the healthcare practitioner to understand what the important information is they should be monitoring to allow them to become empowered.

Alleviating manual demand on the patient:
The present invention alleviates manual demands on the patent by reducing the typical 39 steps required in CAPD to 27. Differentiating the actual PD exchange into two phases is also critically important. The preparation phase is one that has a series of infection control points of increasing importance. However the actual PD exchange phase is one where patient interaction is limited to only a few interactions thereby reducing the complexity and challenge of not making a mistake and thereby decreasing the burden of therapy for the patient.

The weigh scales 24, 26 for bags 20, 22 of dialysate infused and drained can use analogue load cells, but the analogue signals can be converted into digital signals or digital pressure sensors can be used. As there is also the possibility that the specific gravity of the various solutions being measured may vary, the ability to provide modified volume calculations are also provided by the system.

The system according to the present invention not only minimises the manual steps required, but allows for ‘catch-up’ of non-recorded information, such as when a patient may have performed a manual exchange without using the system or device 10 of the present invention.

The information captured effortlessly in the present invention can become important for the management of the patient.

Gravity based infusion system:

A critical technical achievement of the present invention is that the power board system on the device 10 (in the tablet 28) uses time/date-stamp heartbeat and this is set to synchronise with the greater CAPD system 50 when the patient initiates the PD treatment on their user interface (tablet 28) and more specifically the signal originating from the CAPD management application 44 ensuring viable and reliable data for each CAPD dialysis session performed by the patient.
The user interface (tablet 28) and the device 10 are also uniquely paired and a process to confirm the actual identity of the user interface (tablet 28) and the dialysis device 10 in an environment like a medical ward where there may be more than one patient using a dialysis device, prevents communication with the wrong device.
Similarly in a family home environment where the cause of ESRD is familial and there are two or more patients performing dialysis, the unique pairing can prevent the cross contamination of data from device 10 to user interface (tablet 28).
While the system utilises gravity and the Intraperitoneal Pressure (IPP) of the patient as the pressure forces to drive infusion and draining of dialysate, the interaction of these forces allows for individualised calculations to be made that are dynamic by their nature and allow for individual knowledge of each patient individually

There are a host of safety features built into the gravity assisted dialysis device procedure:
Infection control points directly ensure that the patient has performed those essential activities that are defined by international guidelines of treatment or by a protocol from a specific medical team that is responsible for the treatment of the patient.
There are critical safety points that are not only important for patient safety but for the well-being of the patient.
Critical safety points—if the device loses control of the CAPD procedure an emergency feature is triggered which asks the patient to physically stop or shut the device down to prevent harm. The patient flow controlling devices, i.e. the clamps 24, 26 are also designed such that when there is a loss of power to the motor of either or both clamps, they will by their nature of design close automatically. Furthermore to reduce undue strain on the mechanical designs of the clamps, when they are either fully opened, or fully closed, they do not require excessive mechanical force to remain in those positions.
There are also various verification steps to ensure that the electronic circuitry is actually recording as is being shown on the reading in the user interface in the form of a dialysis treatment application (patient experience module) either to be validated by the patient or by electronic feedback mechanisms. In the event the specific component of the device needs to be reset the patient can initiate this step and the power control console of the device would activate such a procedure.
There are pressure tests and leak tests conducted at critical stages of the treatment that ensure the patient is closing his/her catheter and not exposing him/herself to dangerous situations, or the device is not operating within the required specifications.
There are performance logs to identify when patients make ‘little mistakes’ which individually are not significant but building these up over time allow the medical team to identify a patient starting to struggle to cope with their burden of therapy.
There are also indicators of specific physical measurements of the patient that are made and while the individual measurement may well be within the tolerance of the attending physician, there may be a progressive increase or decrease of these measurements that allow the interpretation to be made that the patient is starting to develop an unfavourable health condition.

There are actual physical steps that must be done which are now simplified using the user interface (tablet 28):
The significance of this is that the patient is not left alone to do the treatment but has easy resources for guidance.
The accumulated stress that occurs for the patient when he/she needs to perform numerous sequential activities and certain of them may lead to unfavourable health conditions is a cause of patient “burn-out” and by changing the emphasis of what the patient needs to perform reduces the stress and the accumulated stress dramatically.
If a patient is unsure, he/she can refer to the training and support materials.
They can reach out to a helpline dedicated to them as in the technology enabled care type mould of a 24/7 assistance.
In countries where resources are insufficient to provide 24/7 support, as an example in Spain the medical team may only work half-day and no other specially trained nurse is available to support a patient, the technology enabled care type call centre is trained and equipped to provide the support as they would have the treatment protocol and any escalation protocol of notification present.
During the critical steps of treatment that today a patient needs to physically manage themselves is now not required.
Additionally they do not need to accurately know how much fluid is drained, or whether the filling solution has stopped as they are now helped if these critical treatment steps by the procedure being done for them.
they have no way to know the fluid flow rate of the draining fluid or the filling fluid, the patient also does not need to monitor if their drain flow rate is not suitable
They need to open and close clamps during their actual treatment and the performance of the device now takes this burden away from them and manages these steps.
Especially unique to each patient, is whether the volume needed to fill at every dialysis treatment is less than the volume in the commercially available solution bag, because the patient may have a smaller body surface area, the patient is not under stress to prevent overfilling him/herself as the volume of the prescription is strictly infused by the device.
A child is currently considered unsuitable to perform CAPD based on the great risk of overfill volume that may be dangerous, but with the present invention, children can now perform CAPD safely.
If a patient is hospitalised and the medical staff is not familiar with the CAPD treatment required, then the medical staff can now perform the dialysis safely as it is possible to have a care-giver function activated which guides the attending medical staff to perform the steps sequentially.
The device 10 checks with the patient at the end of each exchange of the drain or bottom bag 22 and allows instant communication of any potential side-effects. This allows the medical staff to intervene sooner than is experienced in the prior art to initiate treatment for side effects—most notably peritonitis.
If there are repeated slow drain catheter complications identified, the incidence of total mechanical failure of the catheter may be prevented.
Identifying flow insufficiency soon after a patient has started treatment, identifies potential complications that were caused by the surgical procedure to insert the catheter

The accumulated results of these innovations of the present invention are to reduce the stress patients may experience and reduce their burden of treatment.

The present invention includes a dedicated ‘observer’ system in place to allow the efficient enrolling and management by medical professionals of the patients using a remote patient care module, the treatment coordination application to coordinate all their medically based treatments and then the dialysis experience module (user/patient interface) to care for their specific dialysis treatment. The dialysis device communicates with the dialysis experience module. Other medical devices that may be needed for a patient treatment are coordinated with the treatment coordination application.
This is a unique CAPD care environment that is tailored around the patient's lifestyle helping them cope.
They can remain economically active which is very important because their medical condition can place a substantial financial burden on them and their families.
The invention enables the medical team to have vital information they need to manage the patient better.
The invention caters for challenges of the patients' built environments such as living in developing countries where they don't have reliable and efficient electricity by having sufficient power stored in the device should there be a power outage.
The invention caters for patients' not having live constant internet access or expensive data access, by allowing packets of data to be sent when convenient.
The invention does not use excessively costly electronic solutions but can operate using the most affordable communications devices available on the market, such as conventional tablets 28.
The invention caters for the patients' lifestyles if they live in rural communities or if they are urban based but spend their free time rurally by allowing their device to operate off a commercially available 12Volt ‘portable’ battery

Prescription choice and flexibility:
The invention allows patients to adjust their solutions as they have been trained by the medical professionals and as their condition demands or based on the outcome of their previous dialysis treatment.
The invention also allows patients to perform treatments they are aware are normally conducted at a later or earlier time of the day as they may have an exceptional event planned for that specific day.
The invention does not allow the patient to adjust the fill volumes but measures accurately the amount that has been determined as ideal for his/her dialysis treatment by the medical professionals.
The invention has the ability to ensure that the long dwell solution is the one they need and have available to optimise their treatment outcomes.
The invention provides medical professionals with insight into how a patient is utilising the desired prescription practically.
The ‘remote control’ type attempt to manage the patient has always had the challenge that the ‘remote control’ did not reach the patient when and where it was needed. This essentially resulted in the treatment being performed in the dark.

Analytical tools:
The invention allows for customisable dashboards on the treatment coordination application to allow the medical team to manage their patient group and each patient as they need.
The invention allows a patient access to his/her own patient information portal on the dialysis experience module, empowering him/her to be part of the treatment solution if he/she so chooses.
The invention allows the treatment of a patient as a whole, this is done by allowing importation of other disease information that brings together their information if it is deemed important.
The requirement of regulatory authorities is demanding of medical device companies to provide real world data on how the specific medical device is performing to achieve patient safety and achieve patient outcomes. The design of the anonymous event logs of the devices will be a way to achieve this most important new regulatory requirement that is potentially having a major impact on the cost of supporting devices in the marketplace.
The invention protects the patient's rights of privacy of data.
The invention uses a number of algorithms that have been developed to address the specific needs of CAPD Patients.
The invention allows for the standardised formulas that are used under the influence of different global, regional or national centres of expertise, to be modified as they are required.

The system according to the invention measures various factors, for instance:
UF per PD Exchange, which in itself is not a specific definable treatment outcome but calculated over 24 hrs and over comparative time segments can be significant.
Optimising UF outputs by prescription adjustment is known to have long-term impact on technique survival.
The invention provides a solution that assists the medical team to manage their patients, attempts to identify and measure many of the variables, as well as provide defined notifications of out-of-limits parameters.

### Privacy and Information Management

Personal Information of the patient is only created and available to the medical professionals directly responsible for the care of the patient, which resides in the remote patient care module under their control.
All treatment related data communicated from the user interface (tablet 28) and generated by the medical device system, and subsequently returned, are anonymised and encrypted according to international standards.
No personal information is transmitted to the medical devices save for a double authenticated username, which username is the patient's discretion.
Limited personal information (e.g. name, phone number, address and next of kin details) is also made available to technology enable care (TEC) emergency response organisations, who may be contracted to provide after-hours support to patients. Specific details of the emergency they are reacting to are also provided (e.g. blood pressure exceeding levels established by the supervising physician) in order to ensure the protocol to enable the appropriate emergency response, such as hospitalisation, are implemented. The technical support also provided via the contracted TEC provider will have the limited information available to ensure support for device use or to replace a defective device.

Patient outcomes optimisation:
The present invention allows the complex management of patients with ESRD to be used to improve patient outcomes by evaluating the interactive nature all major parameters of data. The primary cause of ESRD may today be one of 285 different causes which all need to be verified by diagnostic criteria. The additional complexity of the possibility that there may be secondary causes of the ESRD are also well established. The treatment of the disease may also vary according to various well-defined criteria that are today evaluated with the experience of the attending health care practitioner. Such examples are the model of kinetics within the abdomen and the characteristics of the peritoneal membrane, such as the (Peritoneal Equilibration Test) PET results and the categorisation of patients relative to their designated ‘Transport Status’. The amount of existing kidney function and generation of daily urine volume may another one.

The coordinator logic of the present invention includes a prescription algorithm that is able to use extensive data of patient cohorts using the invention, to analyse and assess the expected outcomes of treatment against the actual measured outcomes.

These modelled outcomes can be used to provide decision support to healthcare practitioners and constantly review the actual outcome based on all the parameters being measured. This is done by evaluating the data generated and aggregated according to various expected outcomes and outcomes generated in hegemonies communities.

### Online PET Evaluation

Today the PET results and classification of patient peritoneal transported status are considered as an important components of prescriptions specific to a patient. The present invention allows accurate data to be used in the PET test and also aggregates the important patient data required. Pathology results of certain effluent can also be added to provide the PET status. The coordinator logic of the present invention includes an algorithm to evaluate the efficacy of the PET model against expected and actual outcomes.

### Hypertensive Treatment Optimisation

Healthy kidneys provide long term maintenance of blood pressure control and renal replacement therapy also strives to achieve this goal. The complexity of managing ESRD patients' hypertension is expansive and is generally managed pharmacologically and also by using the PD prescriptions to ensure ultrafiltration is efficient. The coordinator logic of the present invention includes logic that uses the data obtained from CAPD to provide decision making support in the management of the patient hypertension, comparing outcomes and prescription data. This model of hypertensive management can be deployed in general management of patients

### Patient Prescription Optimiser Model

PD outcomes have been improving over time but with great variability, driven by individual and system-level inequities and by centre effects. This variation is exacerbated by a lack of standardised outcome definitions. Potential strategies for outcome improvement include enhanced standardisation, monitoring and reporting of PD outcomes, and the implementation of continuous quality improvement programmes and of PD-specific interventions, such as incremental PD, the use of biocompatible PD solutions and remote PD monitoring.

The present invention addresses the challenges of providing outcomes with aggregating of data to ensure that relevant outcome reporting is provided but also then supports decision making. As example the coordinator logic of the present invention includes logic that performs PD exchange fluid rate optimization and that allows for the measurement of the fluid flow rates experienced by patient's to be aggregated and allow for adjustment. As example the flow rate may create painful abdominal episodes, which can now be alleviated by adjusting the flow rate.

The present invention meets or ameliorates an unmet need to allow management of the complex variables of CAPD to allow decision making and to leverage the population of global patients and achieve patient-centric outcomes that is constantly updating and evolving.

### Online Health-Economic Modelling

Health-economic data for the benefit of multiple perspectives is generally very difficult to generate and invariably may cause controversy. However, data aggregation in the present invention allows for health-economic data to be provided in the health-economic stakeholder perspective that is required. As example Quality Adjusted Life Year (QALY) type assessments can be performed easily on the data aggregated in the present invention and the analysis of the outcomes provides efficient feedback for health-economic reporting tools.

### Experimental Study

Background: PD is one of the major renal replacement modalities for ESRD patients. Heart failure is a common adverse event among PD patients, especially for those who operate CAPD at home because of the lack of professional input-output volume monitoring and management during treatment.

Objective: This study aimed to develop novel mobile health (mHealth) tools to improve the quality of home-based CAPD treatment, and build a prediction model of heart failure based on the systems' daily treatment monitoring data.

Methods: The mHealth tools with a four-layer system were designed and developed using Spring Boot, MyBatis Plus, MySQL and Redis as back-end technology stack, and Vue, Element UI and Wechat Mini Program as front-end technology stack. Patients were recruited to use the mHealth tool during daily PD treatment from Jan. 1, 2017 to Apr. 20, 2023. Logistic regression model based on the real-time treatment monitoring data were used for heart failure prediction. Sensitivity, specificity, accuracy and Youden index were calculated to evaluate the performance of the prediction model.

Results: A WeChat mini program named Futou Bao for patients and a patients' data management platform for doctors were developed. The Futou Bao software included an intelligent data upload function module and auxiliary function module. Four function modules made up the doctor's data management platform, including patient management, data visualization and marking, data statistics and system management. During the study period, a total of 6635 peritoneal dialysis patients' records were uploaded by Futou Bao with 0.71% of them (47 patients) experiencing heart failure. The prediction model that included sex, age and diastolic blood pressure was considered as the optimal model, of which the sensitivity, specificity, accuracy and Youden index were 0.75, 0.91, 0.89 and 0.66, respectively, with an area under the curve (AUC) value of 0.879(95% Cl: 0.772-0.986) using the validation dataset.

Conclusions: This study provided a new home-based peritoneal dialysis management paradigm that realized the real-time monitoring and early warning of heart failure risk. This novel paradigm was of great value to improve the efficiency, security and personalization of peritoneal dialysis. The study demonstrated that the present invention meets a substantial, previously unmet need for a modelling technique that uses dynamic data, instead of only static data.

1. Apparatus for performing continuous ambulatory peritoneal dialysis (CAPD) for a patient, said apparatus comprising:
programmable circuitry on which a healthcare provider can enter prescription data;
a user interface that is configured for the patient to enter data and to provide information to the patient, said information including at least sequential instructions to the patient to perform the CAPD treatment according to the prescription data;
instruments for gathering treatment data including at least one of: dates and times when CAPD treatments are preformed; durations of flushing, filling and/or draining; quantities of dialysate solution used during flushing, filling and/or draining; and intraperitoneal hydrostatic pressure (IPP);
a communication module configured to communicate the treatment data to the healthcare provider.

2. The apparatus according to claim 1, wherein the instruments for gathering treatment data include at least one weigh scale that is configured to weigh a dialysate solution container.

3. The apparatus according to claim 2, wherein said weigh scale includes an analogue load cell and circuitry configured to convert an analogue signal emitted from the load cell to a digital signal.

4. The apparatus according to claim 2, wherein the programmable circuitry is configured to convert variations in weight of said dialysate solution container to the quantity of dialysate solution used during flushing, filling and/or draining, using the specific gravity of the dialysate solution.

5-6. (canceled)

7. The apparatus according to claim 1, in which the communication module is remote from the instruments for gathering treatment data, and the apparatus includes circuitry configured to time/date stamp the treatment data.

8. The apparatus according to claim 1, wherein the user interface is configured to obtain feedback that the CAPD treatment is being administered correctly and to log mistakes made by the patient, to provide an indication of the patient's ability to cope with the burden of treatment.

9. (canceled)

10. The apparatus according to claim 1, wherein the instruments for gathering treatment data include at least one flow management constrictor that is configured to measure the type of dialysate solution and concentration of said dialysate solution, said flow management constrictor containing photometric instruments and circuitry configured to convert chemical and physical properties of the dialysate solution to digital information.

11-12. (canceled)

13. A system for performing comprehensive management of continuous ambulatory peritoneal dialysis (CAPD) for a patient, said system comprising apparatus according to claim 1, wherein the healthcare provider is remote from the apparatus for performing CAPD and the communication module is configured to communicate the treatment data to the healthcare provider, remotely.

14. A system according to claim 13, wherein the communication module is configured to allow the healthcare provider to enter prescription data remotely on the programmable circuitry and to communicate the treatment data to the healthcare provider in real time.

15-16. (canceled)

17. A method of managing continuous ambulatory peritoneal dialysis (CAPD) of a person in need thereof, said method comprising:
entering prescription data on programmable circuitry;
generating interactive information and sequential instructions in the programmable circuitry, said interactive information and sequential instructions being for the performance of CAPD treatment according to the prescription data;
displaying the interactive information and sequential instructions on a user interface;
gathering treatment data including at least one of: dates and times when CAPD treatments are preformed; durations of flushing, filling and/or draining; quantities of a dialysate solution used during flushing, filling and/or draining; and intraperitoneal hydrostatic pressure (IPP); and
communicating the treatment data to a healthcare provider via a communication module.

18. The method according to claim 17, in which the step of gathering treatment data includes weighing at least one dialysate solution container on a weigh scale.

19. (canceled)

20. The method according to claim 18, which includes converting variations in the weight of the dialysate solution container to quantity of the dialysate solution, using the specific gravity of the dialysate solution.

21. (canceled)

22. The method according to claim 18, which includes converting a flow rate of the dialysate solution to IPP.

23. The method according to claim 17, in which the communication module is remote from locations where the CAPD treatments are performed and the treatment data are gathered, said method including time/date stamping the treatment data, which includes obtaining feedback from the person performing CAPD treatment via the user interface, that the CAPD treatments being administered correctly.

24-25. (canceled)

26. The method according to claim 17, which includes measuring a type and a concentration of the dialysate solution by converting chemical and physical properties of the dialysate solution to digital information using photometric instruments and circuitry on at least one flow management constrictor.

27. (canceled)

28. The method according to claim 26, which includes verifying that the nature of a dialysate solution accords with the feedback provided by the person in need of CAPD treatment on the user interface.

29. The method according to claim 17, which further includes:
aggregating the treatment data gathered for multiple said persons in need of CAPD, said treatment data including multiple parameters;
gathering and aggregating outcomes data for said persons; and
modelling expected likely outcomes of CAPD treatment as a function of said multiple parameters of treatment data, by reviewing said treatment data and outcomes data.

30. The method according to claim 29, which includes determining PET statuses for each of the persons in need of CAPD, and evaluating efficacy of PET against expected outcomes of CAPD.

31. The method according to claim 29, which includes aggregating blood pressure data from said multiple persons in need of CAPD and modelling blood pressure expected as a function of said multiple parameters.

32. The method according to claim 29, which includes determining an optimised prescription for a person in need of CAPD.

33. (canceled)

34. The method according to claim 29, which includes assessing a combination of measurements that in isolation may not provide interpretation of meaning however when considered together may provide invaluable information directly impacting response to the information.

35. (canceled)
